(19) United States
(12) Patent Application Publication
(10) Pub. No.: US 2008/0056938 A1
(43) Pub. Date: Mar. 6, 2008

(54) FRESHNESS MAINTAINING APPARATUS

(75) Inventors: Wan Taek HAN, Hwaseong-si (KR); Soo Bong HEO, Yongin-si (KR); Seong Gu KIM, Pyeongtaek-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/777,696

(22) Filed: Jul. 13, 2007

(30) Foreign Application Priority Data
Aug. 29, 2006 (KR) 10-2006-0082482

(51) Int. Cl. A23L 3/32; A61L 2/03; B01J 19/08

(52) U.S. Cl. 422/22; 422/243

(57) ABSTRACT

Disclosed herein is a freshness maintaining apparatus for maintaining freshness of an object in the apparatus. The apparatus comprises a first shelf having a first electrode disposed therein a second shelf having a second electrode disposed therein; the first shelf and the second shelf being disposed in a manner to be parallel to one another; and a power supply unit in electrical communication with the first electrode and the second electrode; the first electrode having an electrical polarity that is opposedly disposed to an electrical polarity of the second electrode.

### CROSS-REFERENCE TO RELATED APPLICATION

This application claims the benefit of Korean Patent Application No. 10-2006-0082482, filed on Aug. 29, 2006, in the Korean Intellectual Property Office, the disclosure of which is hereby incorporated by reference in its entirety.

### BACKGROUND OF THE INVENTION

1. Field of the Invention

The present invention relates to a freshness maintaining apparatus. More particularly, the present invention relates to a freshness maintaining apparatus having a plurality of electrodes, the plurality of electrodes being formed by individual electrodes that have an alternating electric polarity between them.

2. Description of Related Art

Microorganisms play an important role in our lives. Some of them cause infectious diseases to a living being, while others prevent diseases. Microorganisms can infect a living being from the air, from water, from animals, from foods and the like, and it is therefore important to effectively control the presence of such microorganisms in order to facilitate an improvement in the quality of human life. Controlling such microorganisms is useful in various fields of industry and various methods have been developed in various fields to do so. However, currently available methods are not applicable to all of the various types of microorganisms, therefore a new method to control all types of microorganisms, especially those present in food, is desired.

Depending upon the types of food, various methods are used to control the microorganisms present in the food and thereby extend expiry dates of the food.

The cell membrane of all organisms comprises free ions, e.g., K⁺, Na⁺, Cl⁻, Ca²⁺. The free ions function as follows: i) controls volume of the cell by generating an osmotic pressure that controls the entrance and the amount of water into the cell; ii) plays a key role in other metabolic processes, such as a transduction process; iii) generates a strong electric field of 10⁷ V/m between the cell membranes. An ion flux via the cell membrane is generated by a concentration of free ions that exists within the cell membrane and the application of a voltage to the cell.

The difference in the electrical potential across the cell membrane of an organism is due to the sum of contributions of all free ions present in the cell. When an external electrical field is supplied to the organism, two possible results may occur. First, when the external electric field is static, a polarization in the cell has a predetermined direction and a size, and when the external electric field oscillates, the free ions are forced to vibrate. Second, when the external electric field is harmonic or alternating, the external electric field functions as a periodical force not only on all ions present in a plasma membrane of the organism but also on all ions present in a protein channel present in the organism. The alternating external electrical field promotes all free ions to vibrate. When an amplitude of the oscillation of the ions is greater than a predetermined threshold, the oscillating ions may give an erroneous signal of “open and close signal” of the protein channel, i.e., a voltage channel. This phenomenon may disrupt an electrochemical balance of the cell membrane, which subsequently may hinder the entire function of the cell.

The method of maintaining of food freshness using an electric field is not popular because of a number of problems one of which is the lack of reproducibility. Maintaining food freshness by using an alternating electric field that destroys undesirable living organisms is useful in refrigeration devices that need to preserve food for long periods of time. However, the lack of reproducibility is an undesirable hindrance to achieving such long term food preservation. FIGS. 1 and 2 illustrate a freshness maintaining apparatus according to the prior art, with electric lines of force showing electric field distribution in the apparatus.

Referring to FIG. 1, a prior art apparatus 100 for preserving food comprises an electrode structure a plurality of metal electrodes disposed in a plurality of shelves 110, a power supply unit 120, each shelf 110 having a metal electrode, with the power supply unit 120 forming an electric field by supplying a voltage to the electrode of the shelf. The shelf 110 is also connected to an anode part of the power supply unit 120 by an electrode connector 130 of the shelf 110. A positive charge is distributed on a surface of the metal electrode of the shelf 110, and accordingly, an electric field is formed.

Since all of the shelves are connected to the anode, the positive charge is distributed on the surface of the metal electrode of the shelf 110 as illustrated in the FIG. 2. As described above, in order to make the oscillating ions give an erroneous signal of “open and close signal” for the protein channel, the electrochemical balance of the cell membrane is disrupted. When the amplitude of the oscillation of the ions is greater than the predetermined threshold because of a stronger electric field and greater density of electric lines, it is easy to disrupt the electrochemical balance of the cell membrane. Referring back to FIG. 2, on a top surface of a highest shelf and a bottom surface of a lowest shelf of the prior art apparatus 100, the electric lines of force are so dense that the electric field prevents the microorganisms from proliferating. However, in the case of shelves located between the highest and lowest shelves, the electric lines of force are relatively sparse, as a result of which the prior art apparatus 100 does not fully maintain freshness of food stored therein.

### BRIEF SUMMARY

Disclosed herein is a freshness maintaining apparatus having a plurality of electrodes, wherein each alternating electrode has a polarity that is opposed to that of an immediately neighboring electrode.

In one embodiment, a freshness maintaining apparatus for maintaining freshness of an object in the apparatus, the apparatus comprising a first shelf having a first electrode disposed therein; a second shelf having a second electrode disposed therein; the first shelf and the second shelf being disposed in a manner to be parallel to one another; and a power supply unit in electrical communication with the first electrode and the second electrode; the first electrode having an electrical polarity that is opposedly disposed to an electrical polarity of the second electrode.

Disclosed herein too is a freshness maintaining apparatus, which can disrupt an electrochemical balance of a microorganism by controlling an ion flux in the microorganism using a plurality of electrodes, wherein each alternating electrode has a polarity that is opposed to that of an immediately neighboring electrode. Disclosed herein too is a freshness maintaining apparatus, which can promote a user's safety since direct contact by the user is prevented by covering a metal electrode of the shelves of the freshness maintaining apparatus with an electrically insulating material. In one embodiment, the electrical insulating material can be a dielectric.

Disclosed herein too is a freshness maintaining apparatus comprising a plurality of shelves; each shelf comprising a plate-shaped electrode; a power supply unit in electrical communication with each plate-shaped electrode, wherein each alternating electrode has a polarity that is opposed to that of an immediately neighboring electrode; wherein the power supply unit supplies a voltage that forms a predetermined electric field between the plate-shaped electrodes, and wherein the plurality of shelves are mutually in parallel.

As noted above, when an amplitude of the external electric field is greater than a predetermined amplitude that may have an effect on the function of the cell, it is difficult to maintain the membrane potential, subsequently disrupting an electrochemical balance in the cell and thereby destroying the cell.

An oscillating ion under forced vibration forces a mechanical pressure or a force on a plasma membrane within the cells of the living organism, and has an effect on an open and close mechanism of ion channels, that contain ions such as Ca²⁺. Consequently, irregular gating of the ion channels caused by the application of the external electric field breaks a biochemical balance of the cell membrane, and has a detrimental effect on activity of an entire cell.

The present invention is based on a mechanism that an oscillating or alternating external electric field may have a detrimental effect on a biochemical environment in a cell.

Additional and/or other aspects and advantages will be set forth in part in the description which follows.

### BRIEF DESCRIPTION OF THE DRAWINGS

The above and/or other aspects and advantages of the present invention will become apparent and more readily appreciated from the following detailed description, taken in conjunction with the accompanying drawings of which:

FIG. 1 is a diagram illustrating a prior art freshness maintaining apparatus;

FIG. 2 is a diagram illustrating the electric lines of force showing distribution and a direction of an electric field in a single shelf of the prior art freshness maintaining apparatus depicted in the FIG. 1;

FIG. 3 is an exemplary schematic depiction illustrating a structure of a freshness maintaining apparatus according to an exemplary embodiment described herein;

FIG. 4 is a cross-sectional exemplary depiction illustrating a shelf according to an exemplary embodiment described herein;

FIG. 5 is an exemplary depiction illustrating a structure in which an electrode is electrically connected to a power supply unit in the shelf of FIG. 4;

FIG. 6 is an exemplary depiction illustrating simulation results of a distribution of the electric lines of force showing uniformity and density of an electric field at a point when a voltage is supplied from the power supply unit as described herein; and

FIG. 7A and FIG. 7B are diagrams illustrating a distance controller used to control a distance between the shelves of FIG. 3.

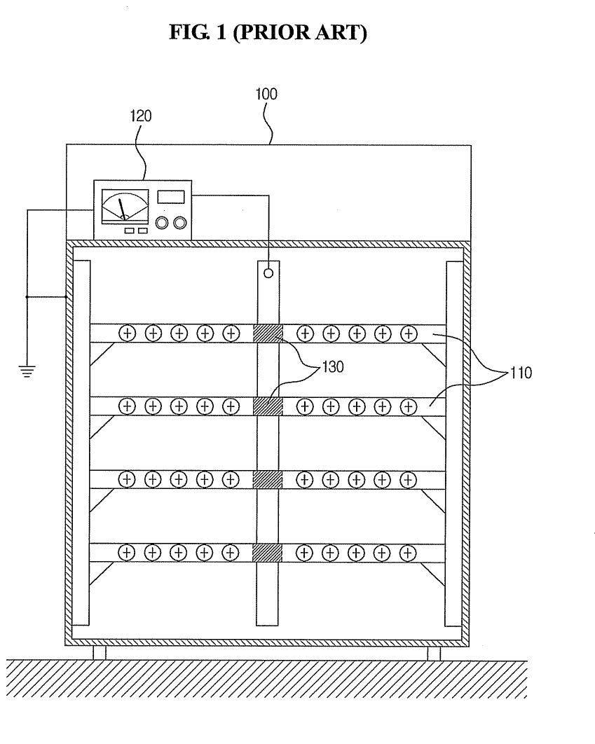

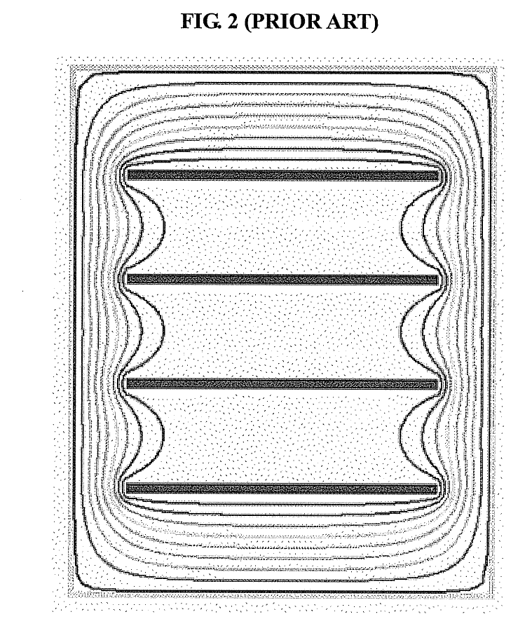

### DETAILED DESCRIPTION OF EMBODIMENTS

Reference will now be made in detail to exemplary embodiments, examples of which are illustrated in the accompanying drawings, wherein like reference numerals refer to the like elements throughout. The exemplary embodiments are described below in order to explain the apparatus and the method described herein by referring to the figures.

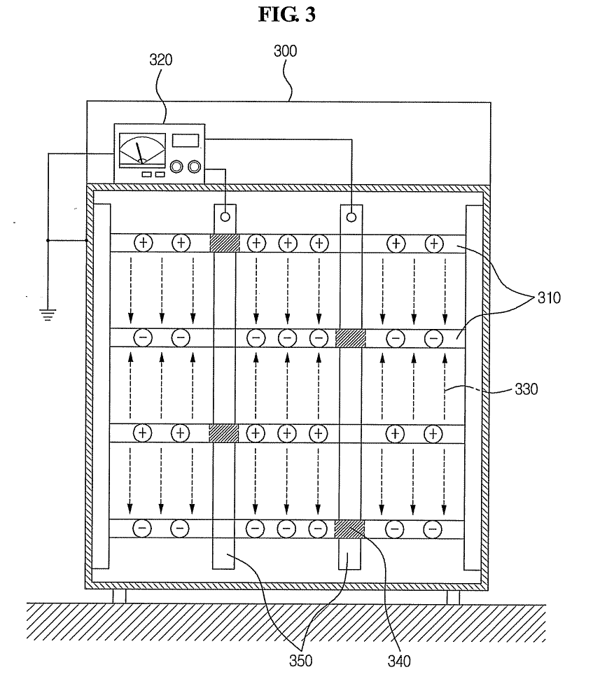

FIG. 3 is a diagram illustrating a structure of a freshness maintaining apparatus according to an exemplary embodiment. Referring to FIG. 3, the freshness maintaining apparatus 300 includes a plurality of shelves 310 and a power supply unit 320.

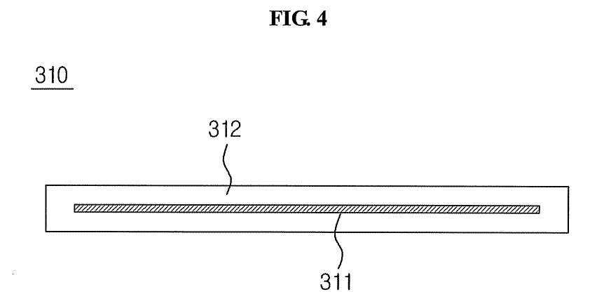

The plurality of shelves 310 each contain an electrode (illustrated in FIG. 4). An object whose preservation is desired may be located on the plurality of shelves 310. As shown in the FIG. 3, in an exemplary embodiment, the surface of the electrode can be flat. The electrode may have at least one surface that has a cross-sectional geometry that is square, rectangular, triangular, circular, or polygonal. Also, the plurality of shelves 310 are parallel with each other.

The power supply unit 320 is in electrical communication with the plate-shaped electrodes disposed within each shelf 310. As noted above, the power supply unit 320 supplies a current of alternating electric polarity to the adjacent plate-shaped electrodes. In other words, as can be seen in the FIG. 3, when the power supply unit 320 supplies current having a positive polarity to a first electrode disposed within an first shelf 310, it supplies current having a negative polarity to a second electrode disposed within a second shelf 310 that is situated adjacent to the first electrode 310, while at the same time supplying current having a positive polarity to a third electrode disposed within a third shelf 310. Thus alternating electrodes in the freshness maintaining apparatus are supplied with electrical currents having an opposing polarity. As can be seen in the FIG. 3, the first and the third shelves are disposed on opposing faces of the second shelf.

When an alternating current is supplied to the electrodes, the alternating current promotes a reversal of polarity of the electrodes. After a while, the power supply unit supplies current having a negative polarity to a first electrode disposed in the first shelf 310, and supplies current having a positive polarity to a second electrode disposed in the second shelf that is disposed adjacent to the first electrode, while at the same time supplying current having a negative polarity to a third electrode disposed in the third shelf. Thus the first electrode and the third electrode alternate between having a positive polarity and a negative polarity, while at the same time, the second electrode alternates between having a negative polarity and a positive polarity.

In addition, the power supply unit 320 may supply a voltage, which is synthesized by at least one of a pulse voltage, an alternating current (AC) voltage and direct current (DC) voltage. The alternating electric field 330 is formed in a vertical direction by supplying a varying voltage.

The electrode in the plurality of shelves 310 is in electrical communication with the power supply unit 320 via the electrode connector 340. As illustrated in the FIG. 3, the plurality of shelves 310 are in parallel with each other and the electrical lines of force between a pair of shelves are opposedly disposed when compared with the electrical lines of force between a neighboring pair of shelves. For example, the electrical lines of force between the first and second shelves are opposedly disposed to the electrical lines of force between the second and third shelves. Consequently, the electric lines of force on a top and a bottom of the shelves 310 are so densely formed so that the ion flux in a microorganism is controlled to cause a disruption in an electrochemical balance of the microorganism. Conversely, electric lines of force on the top and the bottom of the shelves are thinly formed on the prior art freshness maintaining apparatus.

FIG. 4 is a cross-sectional diagram illustrating a shelf 310 according to another exemplary embodiment.

Referring to FIG. 4, the shelf 310 according to the exemplary embodiment includes an electrode 311 and an electrically insulating material 312. The electrically insulating material can be a dielectric. The electrically insulating material generally has an electrical resistivity greater than or equal to about 10¹² ohm-cm.

The electrode 311 forms an electric field between the shelves 310, and is made of and electrically conducting material such as metals, conducting metal oxides or polymers. Examples of metals are gold (Au), silver (Ag), nickel (Ni), chromium (Cr), copper (Cu), or a combination comprising at least one of the foregoing metals. Examples of electrically conducting metal oxides are indium tin oxide (ITO), antimony tin oxide, tin oxide, or a combination comprising at least one of the foregoing electrically conducting metal oxides. Exemplary conducting polymers are polypyrrole, polyaniline, polythiophene, or the like, or a combination comprising at least one of the foregoing conductive polymers. A metal whose tendency to ionize is less than that of the aforementioned metals may be coated on a metal surface of the electrode 311.

The dielectric material 312 prevents the electrode 311 from making direct contact with an object in the apparatus, and is for electrical insulation from an external environment. Examples of electrically insulating dielectric materials are glass, alumina, teflon, TiO₂, BaTiO₃, polyimide, polystyrene, polymethylmethacrylate (PMMA), polyvinylalcohol, polycarbonate, polyolefins such as polyethylene and polypropylene; polyesters such as polyethylene terephthalate and polybutylene terephthalate; polyvinylphenol, benzocyclobutene(BCB), parylene-C, 2-amino-4,5-imidazoledicarbonitrile, metal phthalocyanine, LiF, silicon dioxide, a silicon nitride or derivatives thereof, aluminum oxide (Al₂O₃), Ta₂O₅, AlN, AlON, La₂O₅, BaZrTiO₃, PbZrTiO₃, and an inorganic selected from derivatives of any one of the above inorganic compounds. The shelves of the present invention are not limited to those illustrated in FIG. 4, and a structure made of the dielectric material or a material surrounded by the dielectric material capable of electrical insulation, are sufficient for a structure for the shelves.

The encapsulation of the electrode by an electrically insulating material promotes the user's safety since direct contact by the user is prevented.

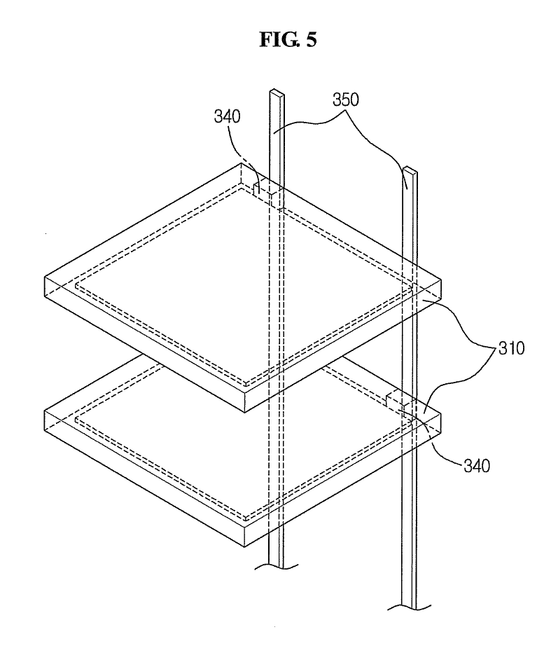

FIG. 5 is a diagram illustrating a structure in which the electrode 311 is electrically connected to the power supply unit 320 in the shelves 310 of FIG. 3. Referring to FIG. 5, a power supply connector 350 of the power supply unit 320 is in electrical communication with the electrode connector 340 of FIG. 3 made of a conductive material. As illustrated in FIG. 4, the electrode 311 of the shelves 310 is in electrical communication with the power supply connector 350, which is also in electrical communication with the power supply unit 320 of FIG. 3. Also, as illustrated in FIG. 5, the shelves 310 of FIG. 3 are alternatingly in electrical communication with the power supply connector 350 via the electrode connector 340 of FIG. 3. Accordingly, an alternating electric polarity of the electrode 311 included in the shelves 310 is formed, and the electric polarity of the electrode 311 of FIG. 3 periodically alternates between positive and negative when a voltage, supplied from the power supply unit 350 of FIG. 3, is an oscillating voltage such as an AC voltage.

As described above, when the oscillating voltage is harmonic or alternating, an external electric field functions as a periodical force not only on all ions in a plasma membrane but also on all ions in a protein channel. The external periodical force forces all ions to vibrate. When an amplitude of the oscillation of the ions is greater than a predetermined threshold, the oscillating ions may give an erroneous signal of “open and close signal” of the protein channel, i.e. a voltage channel. It is desired that the oscillating voltage is less than 10 kV considering ozone between the shelves 310. This phenomenon disrupts an electrochemical balance of the cell membrane, subsequently hinders the entire function of the cell.

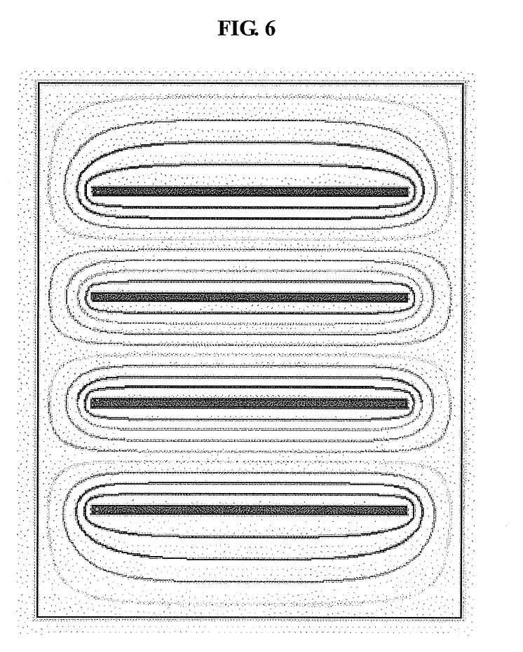

FIG. 6 is a diagram illustrating simulation results of a distribution of the electric lines of force showing uniformity and density of an electric field at a point when a voltage is supplied from the power supply unit according to an exemplary embodiment. Referring to FIG. 6, the electric lines of force are dense on top of the shelves 310 of FIG. 3 where an object such as food whose preservation is desired is actually located, and uniformity becomes lower and becomes relatively sparse as the electric lines of force near a wall of the freshness maintaining apparatus 300 of FIG. 3, from the shelves 310 of FIG. 3. Thus, according to the freshness maintaining apparatus 300 of FIG. 3, an ion flux in the microorganism may be easily controlled using the electrode formed with the alternating electric polarity.

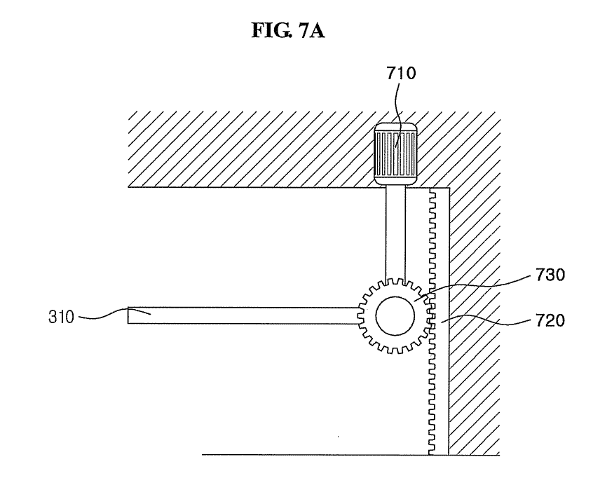

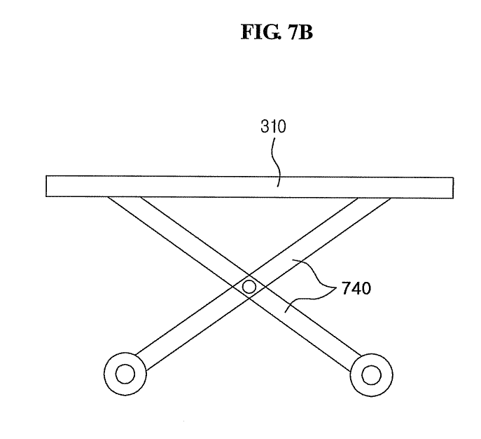

FIG. 7A and FIG. 7B are diagrams illustrating a distance controller to control a distance between the shelves 310 of FIG. 3.

Referring to FIG. 7A, the distance controller according to the exemplary embodiment of the present invention includes a motor 710, a rack 720 and a pinion 730. A distance between the shelves 310 of FIG. 3 may be automatically controlled by connecting the motor 710 to the pinion 730. Namely, when the pinion 730 rotates according to the operation of the motor 710 by connecting the motor 710 to the pinion 730, the rack 720, engaged with the pinion 730, rotates according to the rotation of the pinion 730, and the distance between the shelves 310 of FIG. 3 may be controlled according to the rotation of the rack 720.

In another embodiment, the plurality of electrode units may be configured to automatically ascend or descend by mounting a sensor and measuring a height of an object on a housing member of the maintaining apparatus, and the distance between the electrodes of the multiple electrode units may be controlled since a user controls the distance using a control button without the sensor.

Referring now to FIG. 7B, the distance controller according to another embodiment of the present invention includes a shelf which is supported by an X shaped link 740, the X shaped link 740 has a groove and a protrusion, and a location of the X shaped link 740 may be vertically controlled by a control button since the link is connected to a motor.

According to the present invention, there is provided a freshness maintaining apparatus having an electrode structure, the electrode structure forming an alternating electric polarity of the electrode.

Also, according to the present invention, there is provided a freshness maintaining apparatus, which can break an electrochemical balance of a microorganism by controlling an ion flux in the microorganism using an electrode forming an alternating electric polarity.

Also, according to the present invention, there is provided a freshness maintaining apparatus, which can promote user's safety since direct contact by the user is prevented by covering a metal electrode of shelves of the freshness maintaining apparatus with a dielectric material.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A freshness maintaining apparatus for maintaining freshness of an object in the apparatus, the apparatus comprising:
a first shelf having a first electrode disposed therein;
a second shelf having a second electrode disposed therein; the first shelf and the second shelf being disposed in a manner to be parallel to one another; and
a power supply unit in electrical communication with the first electrode and the second electrode; the first electrode having an electrical polarity that is opposedly disposed to an electrical polarity of the second electrode.

2. The apparatus of claim 1, wherein the first shelf and the second shelf comprise an electrode having a surface whose cross-sectional area is square, rectangular, triangular, or polygonal.

3. The apparatus of claim 1, wherein the first shelf and the second shelf each comprise an electrode that is encapsulated in an electrically insulating material.

4. The apparatus of claim 1, wherein the power supply unit supplies a voltage which is synthesized by a pulse voltage, an alternating current (AC) voltage or a direct current (DC) voltage.

5. The apparatus of claim 3, wherein the electrode comprises an electrically conducting material and wherein the electrically conducting material is gold, silver, nickel, chromium, copper, indium tin oxide, antimony tin oxide, tin oxide, or a combination comprising at least one of the foregoing electrically conducting materials.

6. The apparatus of claim 3, wherein the electrically insulating material is glass, alumina, teflon, TiO₂, BaTiO₃, polyimide, polystyrene, polymethylmethacrylate, polyvinylalcohol, polyvinylphenol, BCB (Benzocyclobutene), perylene-C, 2-amino-4,5-imidazoledicarbonitrile, metal phthalocyanine, LiF, silicon dioxide, silicon nitride or its derivatives, aluminum oxide (Al₂O₃), Ta₂O₅, AlN, AlON, La₂O₅, BaZrTiO₃, PbZrTiO₃, a derivative of any one of the foregoing electrically insulating materials.

7. The apparatus of claim 1, further comprising:
a distance controller to control a distance between the first shelf and the second shelf.

8. A method for maintaining freshness comprising:
disposing an object whose preservation is desired upon a shelf; the shelf comprising an electrode that is in electrical communication with a power supply unit; and
subjecting the electrode and the object to an alternating electrical field.

9. The method of claim 8, further comprising a plurality of shelves, wherein each shelf has an electrical polarity that is opposed to an electrical polarity of its neighboring shelves.
